(19) United States
(12) Patent Application Publication
(10) Pub. No.: US 2005/0267414 A1
(43) Pub. Date: Dec. 1, 2005

(54) Device and method for releasing a therapeutic agent brought into the interior of a patient's body

(76) Inventors: Klaus Abraham-Fuchs, Erlangen (DE); Friedrich Fuchs, Roettenbach (DE); Rainer Kuth, Herzogenaurach (DE); Johannes Reinschke, Nuernberg (DE); Guenter Ries, Erlangen (DE); Rudolf Roeckelein, Erlangen (DE)

Correspondence Address: HARNESS, DICKEY & PIERCE, P.L.C., P.O.BOX 8910, RESTON, VA, 20195, US

(21) Appl. No.: 11/139,631

(22) Filed: May 31, 2005

(30) Foreign Application Priority Data
Jun. 1, 2004 (DE) 10 2004 026 615.8

(51) Int. Cl. A61M 5/00

(52) U.S. Cl. 604/173; 604/93.01

(57) ABSTRACT

For releasing a therapeutic agent, a release is disclosed. The release allows a release or expulsion of the therapeutic agent that is adapted appropriately for the therapy, and/or for the specific properties of the therapeutic agent.

The present application hereby claims priority under 35 U.S.C. §119 on German patent application number DE 10 2004 026 615.8 filed Jun. 1, 2004, the entire contents of which is hereby incorporated herein by reference.

### FIELD

The invention generally relates to a device and/or to a method for treatment or examination of the interior of a patient's body.

### BACKGROUND

For the treatment or examination of the interior of a patient's body, DE 101 42 253 C1 discloses an endorobot, which is provided with a linear magnet and can be navigated in a 3D gradient field into a position suitable for the treatment or examination under remote control without a fixed connection to the outside. The operating head can be equipped with various types of tool arms, with which for example a tissue sample can be taken in the corresponding treatment or examination position.

Furthermore, it is possible to equip the operating head with different measuring sensors for monitoring the temperature or the electrical conductivity and for detecting chemical substances. The operating head is provided in the known case for transporting medicaments and their targeted release at the treatment position, although no details concerning the type of release or the way in which it functions are specified.

### SUMMARY

An embodiment of the present invention includes an object of bringing different therapeutic agents reliably into a position for therapy in the interior of a patient's body, in a simple manner for example. Further, the embodiment includes releasing them there effectively in a way which can be adapted appropriately for the respective therapeutic agent.

The device according to an embodiment of the invention and the method according to an embodiment of the invention make it possible for a therapeutic agent to be transported reliably and released in a way which can be adapted appropriately for the therapy into the diseased tissue of a patient by a device/way of release provided for this purpose, the device/way of release being able to be brought from a prestressing position, fixed before the beginning of examination and secured for transportation, into a defined destressing position, effectively expelling the therapeutic agent, in the position for therapy.

In one refinement of an embodiment of the invention from the aspect of compact installation and at the same time effective release of the therapeutic agent, a prestressed spring element, a plunger or a chemically effective propellant charge is advantegously provided as the means of release. The various devices/ways of release can in this case be secured in a simple way during the movement of the probe in the interior of a patient's body and, in the secured prestressing position, take up only a small overall volume in an operating head assigned to the probe.

By specific selection of one of these devices/ways of release, an expulsion of the therapeutic agent from the probe into the diseased tissue that is optimally adapted appropriately for the therapeutic agent or the therapy is additionally possible. The therapeutic agent can be reliably introduced in a targeted manner into the diseased tissue by a thrust-like movement of the plunger or can be carefully placed in a positionally accurate manner directly on a surface of the diseased tissue by the use of a slowly destressing chemical propellant charge, without the organs or parts of organs that are adjacent to a therapy zone coming into contact with the therapeutic agent.

The triggering or destressing of the device/way of release from the prestressing position into the destressing position and the associated release of the therapeutic agent can, according to one refinement of an embodiment of the invention, take place under remote control, under time control or else under temperature control, so that a release of the therapeutic agent is in each case ensured in the situation in which the best effect of the therapy is achieved.

### BRIEF DESCRIPTION OF THE DRAWINGS

The invention and further advantageous refinements of the invention according to the subclaims are explained in more detail below on the basis of schematically represented exemplary embodiments in the drawings, in which:

FIG. 1 shows an endorobot used as a probe in a side view, with a carrier capsule and an operating head assigned to the latter,

FIG. 2 shows the operating head in a side view, with a first device for releasing a therapeutic agent by a spring element in a prestressing position, provided as a device/way of release,

FIG. 3 shows the operating head according to FIG. 2 in a side view, but with the device/way of release in a destressing position,

FIG. 4 shows the operating head in a side view, with a second device for releasing a therapeutic agent by a chemical propellant charge in a prestressing position, provided as a device/way of release,

FIG. 5 shows the operating head according to FIG. 4 in a side view, with the device/way of release in a destressing position,

FIG. 6 shows the operating head in a side view, with a third device for releasing a therapeutic agent by a spring-driven plunger in a prestressing position, provided as a device/way of release,

FIG. 7 shows the operating head according to FIG. 6 in a side view, with the device/way of release in a destressing position,

FIG. 8 shows the operating head in a side view, with a fourth device for releasing a therapeutic agent by a magnetically driven plunger in a prestressing position, provided as a device/way of release,

FIG. 9 shows the operating head according to FIG. 8 in a side view, with the device/way of release in a destressing position.

### DETAILED DESCRIPTION OF THE EXAMPLE EMBODIMENTS

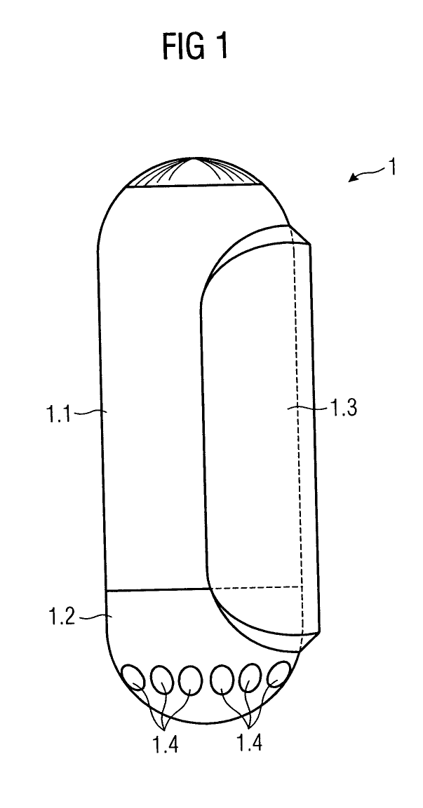

FIG. 1 shows an exemplary embodiment of a probe in the form of an endorobot 1. The endorobot 1 has a carrier capsule 1.1 of a cylindrical shape which extends in the direction of a longitudinal axis and at the end of which an operating head 1.2 is located as part of the carrier capsule 1.1. Arranged over the circumference of the operating head 1.2 are a plurality of outlet openings 1.4, from which a therapeutic agent 4 or 8 to be released can emerge. Fastened to the side of the carrier capsule 1.1 is a magnetic element 1.3, which extends in the direction of the longitudinal axis.

The endorobot 1 can be contactlessly navigated in the interior of a patient's body by way of a 3D gradient field generated in an area under examination. The 3D gradient field can in this case be set from the outside in such a way that, as a result of interactions, forces act on the magnetic element 1.3, and consequently on the endorobot 1, so that the endorobot 1 can be brought specifically into a position for therapy by translatory and rotatory navigational movements.

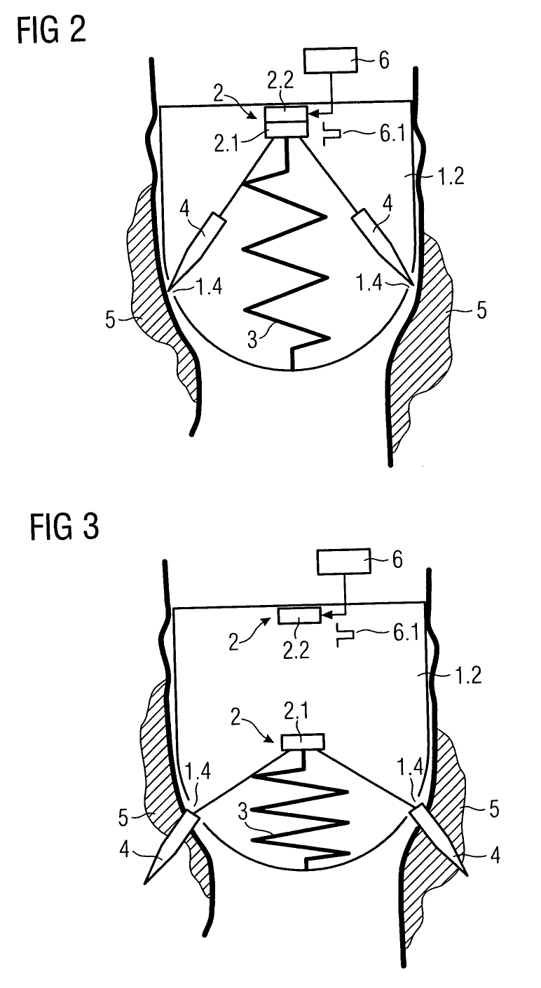

In the operating head 1.2 of the carrier capsule 1.1 there is provided a device for releasing a therapeutic agent 4 or 8, explained in more detail in FIGS. 2 to 9. FIGS. 2, 3 show in a side view, as a first exemplary embodiment, an operating head 1.2 which is brought into the proximity of a tissue 5 and has a release, or device for releasing, in the form of a tension spring 3. This tension spring 3 is represented in FIG. 2 in a secured prestressing position, for transporting the therapeutic agent, and in FIG. 3 in a destressing position, provided for expelling the therapeutic agent.

The device from FIG. 2 has apart from the tension spring 3 that is provided as a device/way of release a stressing device 2, including two parts, and a signal transmitter 6, connected to the stressing device 2. The therapeutic agent to be released has in FIG. 2 the form of needles 4 and is respectively stored directly in front of an outlet opening 1.4 provided on the operating head 1.2.

In the case of the prestressing position of the tension spring 3 that is represented in FIG. 2, its one end is firmly anchored on the head-side end of the operating head 1.2 and the correspondingly other end of the tension spring 3 is secured under stress on the opposite stressing device 2. The stressing device 2 for its part includes two parts; the first stressing part 2.1 is in firm connection with the end of the tension spring 3; the second stressing part 2.2 is attached directly to the operating head 1.2. In the prestressing position shown, the two stressing parts 2.1, 2.2 lie against each other and are also secured against unintentional release of the connection during the transport of the endorobot 1. The release of the connection and the associated destressing of the tension spring 3 for expelling the needles 4 is possible by way of the signal transmitter 6.

The destressing or springing back of the tension spring 3 has the effect that the needles 4 are driven into the surrounding tissue 5 through the outlet openings 1.4 in the operating head 1.2. The device/way of release thereby changes abruptly from the prestressing position shown in FIG. 2 into the destressing position shown in FIG. 3. In the destressed state according to FIG. 3, the needles 4 have completely left the outlet openings 1.4 and are firmly anchored in the surrounding tissue 5.

The expulsion of the therapeutic agent provided in this exemplary embodiment in the form of needles 4 can be optimally adapted appropriately for the therapeutic agent used or for the consistency of the tissue 5 to be therapeutically treated by corresponding choice of the spring constant of the tension spring 3, so that the penetration of the tissue 5 with the therapeutic agent that is most effective for the therapy can be achieved by simple devices/methods.

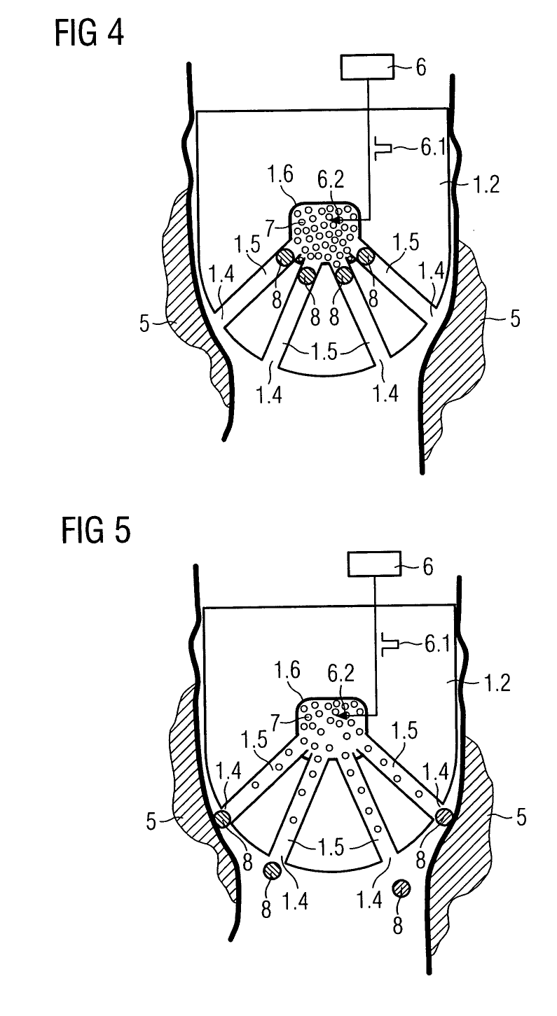

As further variants of a device according to an embodiment of the invention, according to FIGS. 4, 5, a device/way of release is provided in the form of a chemical propellant charge 7, which releases a therapeutic agent in the form of beads 8. The device/way of release is represented in FIG. 4 in a prestressing position and in FIG. 5 in a destressing position, expelling the beads 8.

The device in FIG. 4 has, substantially, a cavity 1.6, which is intended for storing the prestressed chemical propellant charge 7 and is assigned to the operating head 1.2, a fuze 6.2, which is provided in the chemical propellant charge 7, a signal transmitter 6, which is connected to the fuze, and a number of guiding channels 1.5, for the expulsion of the beads 8. Each of the guiding channels 1.5 respectively connects the cavity 1.6 to one of the outlet openings 1.4.

According to FIG. 4, in the prestressing position the beads 8 are arranged at the cavity-side end of the guiding channels 1.5 and, for example as a result of a slight constriction of the guiding channels 1.5 that is not shown any more specifically here, are in frictional connection with the latter, so that on the one hand, in the prestressing position, the beads 8 are secured in a secured transporting position and on the other hand, by destressing the chemical propellant charge 7, the beads 8 can easily be expelled from the holder.

The prestressed chemical propellant charge 7 can be chemically destressed by a signal 6.1 which can be generated by the signal transmitter 6 and can be transmitted to the fuze 6.2, so that the beads 8 are released from their holder by a chemically induced reaction and an associated expansion of the chemical propellant charge 7, are guided along the guiding channels 1.5 through the outlet openings 1.4 and expelled into the diseased tissue 5. FIG. 5 shows the chemical propellant charge 7 from FIG. 4, provided as a means of release, in its destressing position. As a result of a chemical reaction, the chemical propellant charge 7 in the expanded state has escaped into the guiding channels 1.5 in the sense of a pressure equalization with the surroundings, so that, for the therapy of the diseased tissue 5, the beads 8 are lodged on its surface as a reaction to the expansion, as represented in FIG. 5.

The device/way of release in the form of a chemical propellant charge 7 can be particularly easily dimensioned in terms of its effect by simple device/methods by the destressing process or the expansion of the chemical propellant charge 7 being specifically predeterminable by a specific selection of a chemical composition and by an exactly determinable amount of the chemical substances to be used for the chemical propellant charge 7. In this way, depending on the therapy and the form or configuration of the therapeutic agent, a therapeutic agent can be slowly brought up to the diseased tissue 5, or driven into the tissue 5 in an adapted way by a rapid and strong expansion of the chemical propellant charge 7.

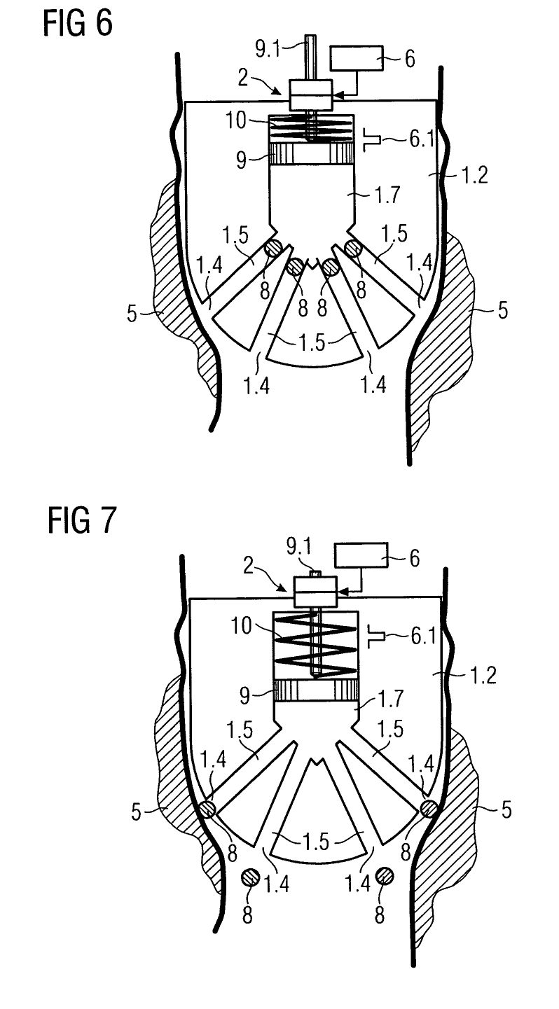

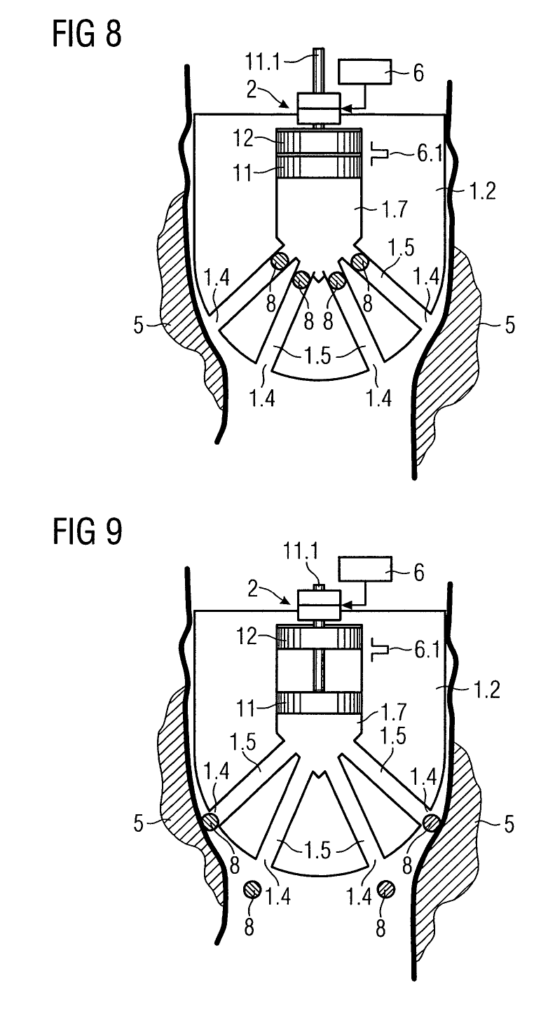

FIGS. 6, 7 and 8, 9 show in comparison with FIGS. 4, 5 two further variants of the device according to an embodiment of the invention, with the difference that, instead of the chemical propellant charge 7, a plunger 9 that is spring-driven by a compression spring 10 is provided in FIGS. 6, 7 and a magnetically driven plunger 11 is provided in FIGS. 8, 9.

In FIG. 6 the spring-driven plunger 9 and in FIG. 8 the magnetically driven plunger 11 is shown in its respective prestressing position. The plungers 9 and 11 are respectively connected to the stressing device 2 by a guiding pin 9.1 and 11.1. The guiding pins 9.1 and 11.1 are located in a position secured with respect to the stressing device 2.

In FIG. 6, a supported compression spring 10 is stressed between the stressing device and the spring-driven plunger 9. The guiding pin 9.1 can be released from its anchorage by a signal 6.1, which can be generated by a signal transmitter 6 and can be transmitted to the stressing device 2, so that the spring-driven plunger 9 is driven forward by a destressing of the compression spring 10 in a plunger chamber 1.7. The resultant compression of a gas or liquid stored in the plunger chamber 1.7 and the pressure building up have the effect that the beads 8 and the gas or liquid are expelled into the tissue 5.

In FIG. 8, the magnetically driven plunger 11 has a magnetic material and is mounted in front of a magnetic element 12 assigned to the stressing device 2. The magnetically driven plunger 11 and the magnetic element 12 have the same magnetic polarity, so that they repel each other. The guiding pin 11.1 can be released from its anchorage by the signal 6.1, which can be generated by the signal transmitter 6 and can be transmitted to the stressing device 2, so that the magnetically driven plunger 11 is driven forward into the plunger chamber 1.7 by its magnetically repelling effect with respect to the magnetic element 12.

FIGS. 7, 9 show the respective plunger in its destressing position. The therapeutic agent in the form of beads 8 is released into the tissue 5.

In an advantageous configuration of an embodiment of the invention, the device/way of release 3 or 7 or 9, 10 or 11, 12 can be destressed under remote control, under temperature control or under time control. The release of the therapeutic agent 4 or 8 can consequently take place in a situation that is optimal for the therapy. In addition, the triggering of the device/way of release 3 may also be controlled by other sensors, which for example measure a pressure, a pH value, electrolytic values of an intestinal secretion, a positional value or else the state of an internal bleeding.

The device/way of release can be used particularly effectively for the release of a therapeutic agent for radiotherapy or hyperthermia in that a defined penetration of the diseased tissue with the therapeutic agent required for the therapy can be achieved. A therapeutic agent equipped with a radioactive emitter for the radiotherapy can be positioned directly on the surface of a diseased tissue 5, for example by a device/way of release in the form of a chemical propellant charge 7, so that the radioactive radiation released for the therapy is released onto the diseased tissue directly near the surface. On the other hand, for example, a therapeutic agent which is intended for hyperthermia, is tuned to resonate with an external alternating electromagnetic field necessary for the therapy and can be spontaneously heated up by an external alternating field, can be introduced completely into the diseased tissue 5 particularly effectively by a device/way of release in the form of a spring-back tension spring 3.

The device/way of release according to an embodiment of the invention is suitable for transporting and expelling very different therapeutic agents. A therapeutic agent in the form of beads 8, as represented for example in FIGS. 4, 5 in conjunction with a device/way of release in the form of a chemical propellant charge 7, is particularly suitable for the treatment of superficial tissue disorders, since a bead naturally has a large surface area in relation to its volume. On the other hand, a therapeutic agent in the form of needles 4, as represented for example in FIGS. 2, 3 in conjunction with a device/way of release in the form of a tension spring 3, is suitable particularly effectively for treatments in which the therapeutic agent must be firmly anchored in the tissue 5 and in which a penetration of the therapeutic agent is required.

Also provided for the local treatment of a tissue in the interior of a patient's body are probes with a therapeutic agent to be released, which include a solid, liquid or gaseous pharmaceutical or contain on their surface or in their cavity a pharmaceutical which is given off into the surrounding tissue by diffusing to the surroundings over a prolonged period of time. Similarly provided is a therapeutic agent which dissolves chemically over a fixed, defined period of time.

An advantageous release of a therapeutic agent 4 or 8 in the interior of a patient's body by way of a probe introduced into the patient in the form of an endorobot 1 and a device/way of release 3 or 7 or 9, 10 or 11, 12 assigned to it is characterized by way of example by method steps of:
a) introducing the probe 1 with a device/way of release 3 or 7 or 9, 10 or 11, 12 located in a prestressing position for the release of the therapeutic agent 4 or 8 into the interior of a patient's body;
b) navigating the probe 1 into the position for therapy;
c) triggering the device/way of release 3 or 7 or 9, 10 or 11, 12 from the prestressing position into the destressing position and thereby expelling the therapeutic agent 4 or 8.

An aspect of an embodiment of the invention can be summarized as follows:

For releasing a therapeutic agent 4 or 8, a device/way of release 3 or 7 or 9, 10 or 11, 12 which ensures a release or expulsion of the therapeutic agent 4 or 8 that is adapted appropriately for the therapy and for the specific properties of the therapeutic agent 4 or 8 is provided.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A device for releasing a therapeutic agent brought into a position for therapy in the interior of a patient's body via a probe, comprising:
a release, assigned to the probe and movable from a prestressing position into a destressing position, for releasing the therapeutic agent.

2. The device as claimed in claim 1, wherein the release includes a prestressed spring element.

3. The device as claimed in claim 1, the release includes a prestressed plunger.

4. The device as claimed in claim 1, the release includes a chemically effective propellant charge.

5. The device as claimed in claim 1, wherein the release is movable from the prestressing position into the destressing position under remote control.

6. The device as claimed in claim 1, wherein the release is movable from the prestressing position into the destressing position under time control.

7. The device as claimed in claim 1, wherein the release is movable from the prestressing position into the destressing position under temperature control.

8. The device as claimed in claim 1, wherein the therapeutic agent is provided in the form of beads.

9. The device claimed in claim 1, wherein the therapeutic agent is provided in the form of needles.

10. The device as claimed in claim 1, wherein the therapeutic agent is provided in the form of at least one of a solid, liquid and gaseous pharmaceutical.

11. The device as claimed in claim 1, wherein the therapeutic agent has a radioactive emitter suitable for local radiotherapy.

12. The device as claimed in claim 1, wherein the therapeutic agent is tuned for local hyperthermia to resonate with an external alternating electro-magnetic field necessary for the therapy.

13. The device as claimed in claim 1, wherein the therapeutic agent has a pharmaceutical which is at least one of provided on a surface and arranged in a cavity of the therapeutic agent and diffuses into the surroundings over a prolonged period of time.

14. The device as claimed in claim 1, wherein the therapeutic agent is chemically soluble over a predeterminable period of time.

15. The device as claimed in claim 1, wherein the probe is an endorobot provided with a magnetic element, navigatable in a magnetic field penetrating an area of the patient that is under.

16. A method for releasing a therapeutic agent brought into a position for therapy in the interior of a patient's body via a probe, the method comprising:
a) introducing the probe with a device, located in a prestressing position, for the release of the therapeutic agent into the interior of a patient's body;
b) navigating the probe into the position for therapy; and
c) triggering the device from the prestressing position into a destressing position, and thereby expelling the therapeutic agent.

17. An apparatus, adapted to perform the method as claimed in claim 16.

18. A probe, including the device of claim 1.

19. A device for releasing a therapeutic agent brought into a position for therapy in the interior of a patient's body via a probe, the device comprising:
means, initially in a prestressing position, for releasing the therapeutic agent into the interior of a patient's body; and
means, upon the probe being in the position for therapy, for triggering the means for releasing from the prestressing position into a destressing position, and thereby expelling the therapeutic agent.

20. A probe, including the device of claim 19.
